(19) United States
(12) Patent Application Publication
(10) Pub. No.: US 2023/0172979 A1
(43) Pub. Date: Jun. 8, 2023

(54) Aptamer-Engineered Natural Killer Cells for Cell-Specific Adaptive Immunotherapy

(71) Applicant: The Methodist Hospital, Houston, TX (US)

(72) Inventor: Youli ZU, Bellaire, TX (US)

(21) Appl. No.: 17/504,497

(22) PCT Filed: Apr. 16, 2020
(86) PCT No.: PCT/US2020/028602

§ 371 Date: Oct. 18, 2021

Related U.S. Application Data
(60) Provisional application No. 62/834,602, filed on Apr. 16, 2019.

(51) Int. Cl. A61K 35/17; C12N 15/115; C12N 5/00; C12N 5/0783; A61P 35/02

(52) U.S. Cl. CPC A61K 35/17; C12N 15/115; C12N 5/0006; C12N 5/0646; A61P 35/02; C12N 2310/16; C12N 2310/3515

(57) ABSTRACT

Disclosed are methods and compositions for diagnosis, imaging, and treating one or more mammalian diseases, including, for example, treatment, prophylaxis, and/or amelioration of one or more symptoms of a human cancer. Disclosed are compositions and methods for the treatment and amelioration of one or more symptoms of a disease, and in exemplary embodiments, for use as an immunotherapeutic.

### CROSS-REFERENCE TO RELATED APPLICATIONS

The present application claims priority to PCT Intl. Pat. Appl. No. PCT/US2020/028602; filed Apr. 16, 2020 (pending), which claims priority to U.S. Prov. Pat. Appl. No. 62/834,602, filed Apr. 16, 2019 (expired), the contents of each of which is specifically incorporated herein in its entirety by express reference thereto.

### STATEMENT REGARDING FEDERALLY SPONSORED RESEARCH OR DEVELOPMENT

None.

### NAMES OF THE PARTIES TO A JOINT RESEARCH AGREEMENT

Not Applicable.

### BACKGROUND OF THE INVENTION

### Field of the Invention

The present disclosure relates to the fields of medicine, molecular biology, and specifically to the area of immunotherapy. Provided are compositions and methods for treatment and amelioration of one or more symptoms of disease, and particularly as an immunotherapeutic. In certain applications, the disclosed aptamer-engineered NK cells (ApEn-NK) may be prepared without genetic alteration or cell damage, providing utility for specifically targeting lymphoma cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the present invention. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, FIG. 1F, FIG. 1G, and FIG. 1H show the generation of cell-specific ApEn-NK cells. FIG. 1A: ApEn-NK formation scheme. FIG. 1B: Aptamer-anchor structures. The CD30-specific ssDNA aptamer sequence was conjugated to different lipophilic anchors, specifically single C18 hydrocarbon chains (Apt-C18), dual C18 hydrocarbon chains (Apt-2×C18), cholesterol (Apt-Chol), or vitamin E (Apt-VitE). For tracking purposes, the aptamer sequence was labeled with fluorochrome Cy3. FIG. 1C: ApEn-NK with surface-anchored Apt-2×C18. Cultured NK92 cells were used for ApEn-NK production. Left: confocal microscopy image of the formed ApEn-NK under light and fluorescent views; Middle and right: time and dose courses of the surface-anchoring of aptamers on NK92 cells detected by flow cytometry. FIG. 1D: Degradation of surface-anchored aptamers by DNase treatment. Left: confocal microscopy image of ApEn-NK post-DNase treatment; Right: changes in surface-anchored aptamers post-DNase treatment by flow cytometry. Parental NK92 cells were used in control experiments. FIG. 1E: ApEn-NK with surface-anchored Apt-Chol. Left: confocal microscopy; Middle and right: time and dose courses of the surface-anchoring of aptamers. FIG. 1F: Degradation of surface-anchored aptamers by DNase. Left: confocal microscopy post-DNase treatment; Right: changes in surface-anchored aptamers post-DNase treatment. Parental NK92 cells were used in control experiments. FIG. 1G: ApEn-NK with Apt-C18. Left: confocal microscopy; Middle and right: time and dose courses of the cell surface-anchoring of aptamers. FIG. 1H: ApEn-NK with Apt-VitE. Left: confocal microscopy; Middle and right: time and dose courses of the surface-anchoring of aptamers;

FIG. 2A, FIG. 2B, FIG. 2C, FIG. 2D, FIG. 2E, FIG. 2F, FIG. 2G, FIG. 2H, FIG. 2I, FIG. 2J, and FIG. 2K show Characterization of ApEn-NK. FIG. 2A and FIG. 2B: Stable ApEn-NK with Apt-2×C18. No changes in surface-anchored aptamers of ApEn-NK cells were detected by flow cytometry analysis (left) or confocal microscopy (right) in 60 min post-ApEn-NK production. FIG. 2E and FIG. 23F: Unstable ApEn-NK with Apt-Chol. Rapid decrease of surface-anchored aptamers (>80% reduction) was detected by flow cytometry in 15 min post-ApEn-NK production (left), and near complete loss was observed by confocal microscopy in 60 min (right). FIG. 2G and FIG. 2H: Stability time course study of ApEn-NK with Apt-2×C18. Quantitative flow cytometry revealed that the ApEn-NK cells were stable and >40% of surface-anchored aptamer signals remained 10 hrs post-ApEn-NK production. FIG. 2D: Cell proliferation assays. No toxicity of Apt-2×C18 structures in NK92 cells was observed in cultures up to 72 hrs. FIG. 2I, FIG. 2J, and FIG. 2K: Cell-binding specificity of the aptamer sequences. Cell binding assays revealed that synthetic aptamers did not react with NK92 cells (FIG. 2I) or CD30-negative U937, Maver-1, or Jeko-1 cells (FIG. 2J); synthetic aptamers only specifically bound to K299, SUDHL1, and HDM2 cells, which express CD30 FIG. 2K: Random ssDNA sequences of the same length were used in negative (−) controls. Data shown are Mean±SD, n=3; Statistical analysis and graph plotting were performed with OriginPro 8.5. Differences between groups were determined by one-way Analysis of variance (ANOVA). p values of *<0.05 and **<0.01 were considered statistically significant; NS, no statistically significant difference;

FIG. 3A, FIG. 3B, FIG. 3C, FIG. 3D, FIG. 3E, FIG. 3F, and FIG. 2G show specific binding of ApEn-NK to lymphoma cells. FIG. 3A: Schematic of specific interactions between ApEn-NK and target cells. FIG. 3B and FIG. 3C: Specific E/T cluster formation of ApEn-NK. Equal amounts of Effector cells (ApEn-NK or parental NK92 cells) and Target cells (CD30-expressing K299 or CD30-negative U937 cells) were mixed and resulting E/T cell clusters were detected. Left: individual cell populations in the cell mixtures were gated by flow cytometry, including single Effector cells (E), single Target cells (T), and the E/T cell clusters via fluorescence emission of red, green, and both signals, respectively. Right: Percentages (%) of E/T clusters in all cell events were calculated. Parental NK92 cells were used as a baseline for background controls. FIG. 3D: Fluorescence microscopy images of E/T cell clusters. ApEn-NK showed the red fluorescent signal of surface-anchored aptamers, and K299 or U937 cells were pre-stained in green fluorescence. FIG. 3E: Time course analysis of E/T cluster formation. K299, SUDHL-1, and HDLM2 cells are CD30-expressing lymphoma cells; U937, Maver-1, and Jeko-1 are CD30-negative control cells. FIG. 3F: Number of total cells per formed E/T cluster. Effector cells (E) per formed E/T cluster. FIG. 3G: Target cells (T) per formed E/T cluster. Data shown are Mean±SD, n=3; Statistical analysis and graph plotting were performed with OriginPro 8.5. Differences between groups were determined by one-way Analysis of variance (ANOVA). p values of *<0.05 and **<0.01 were considered statistically significant; NS, no statistically significant difference;

FIG. 4A, FIG. 4B, FIG. 4C, FIG. 4D, and FIG. E show Specific killing of lymphoma cells by ApEn-NK. FIG. 4A: Schematic of ApEn-NK effects on targeted cells. FIG. 4B: Schematic of cell killing assays. The pre-stained lymphoma or control cells were treated with ApEn-NK or parental NK92 cells. Post incubation, individual cell populations of the mixtures were initially separated and gated by flow cytometry (left). Subsequently, the gated single lymphoma cells were further analyzed for their apoptosis/death rates by cellular staining of Cy5 Annexin V and eFluor 450, respectively (right). FIG. 4D Specific killing of lymphoma cells by ApEn-NK. Apoptotic/dead rates (%) of individual CD30-expressing lymphoma cells (K299, SUDHL1, and HDLM2 cells) at different E/T ratios were detected and shown as mean±SD (upper row). In control experiments, individual CD30-negative cells (U937, Mayer, and Jeko cells) were tested under the same treatment conditions (lower row). Parental NK92 cells were used as baseline background controls. FIG. 4D: Highest killing effect occurred at the lowest E/T ratio. The average mean±SD of ApEn-NK cellular effects on all three lymphoma cell lines was calculated and normalized against baseline resulting from parental NK92 cells. Results from three off-target control cell lines were also shown. Data shown are Mean±SD, n=3; Statistical analysis and graph plotting were performed with OriginPro 8.5. Differences between groups were determined by one-way Analysis of variance (ANOVA). p values of *<0.05 and **<0.01 were considered statistically significant; NS, no statistically significant difference; and

FIG. 5A, FIG. 5B, FIG. 5C, FIG. 5D, FIG. 5E, FIG. 5F, FIG. 5G, and FIG. 5H, Functional study of ApEn-NK derived from primary human NK cells. FIG. 5A: In vitro expansion of primary NK cells. Primary NK cells were isolated from fresh peripheral blood samples from healthy human donors and cultured in vitro. Viable primary NK cells were counted at day 7, 14, and 21, and cell expansion results from three donors are shown. Primary NK cells cultured for 14-21 days were used for ApEn-NK production. FIG. 5B: Confocal microscopy of ApEn-NK. Light view of ApEn-NK derived from primary human NK cells and fluorescent view of cell surface-anchored aptamer signals. FIG. 5C: Time course study of aptamer anchoring reaction. Expanded primary NK cells were incubated with 1 μmol/L Apt-2×C18 at room temperature and cellular signals derived from surface-anchored aptamers were quantified by flow cytometry at different time points as indicated. FIG. 5D: Dose course study of aptamer anchoring reaction. Expanded primary NK cells were treated for 30 minutes with Apt-2×C18 at different concentrations as indicated, and cellular signals derived from surface-anchored aptamers were quantified by flow cytometry. FIG. 5E and FIG. 5F Specific binding of ApEn-NK to lymphoma cells. E/T cell cluster formation was analyzed by flow cytometry. The single target cells (K299 or U937), single effector cells (primary NK or ApEn-NK), and resulting E/T clusters containing both target and effector cells were quantified. In comparison to primary NK cells, ApEn-NK induced a significant high binding rate to K299 cells, but caused little change in binding to off-target U937 cells. FIG. 5G and FIG. 5H: Specific killing of lymphoma cells by ApEn-NK. Target cells (K299 and U937) were treated with ApEn-NK or primary NK cells derived from three healthy donors at different E/T ratios as indicated. Cell killing effects were determined by quantifying apoptosis/death rates of target cells using flow cytometry. In comparison to primary NK cells derived from the same donors, ApEn-NK had significantly higher killing efficacy of lymphoma cells (K299), but little effect on off-target U937 cells. Notably, the highest killing efficacy was observed at the lowest E/T ratio. Differences between groups were determined by one-way Analysis of variance (ANOVA). p values of *<0.05 and **<0.01 were considered statistically significant; NS, no statistically significant difference.

### BRIEF DESCRIPTION OF THE NUCLEIC ACID SEQUENCES

SEQ ID NO:1, as described herein is a CD-30 specific aptamer having the following DNA sequence: 5′-ACTGGGCGAAACAAGTCTATTGACTATGAGC-3′ (SEQ ID NO:1).

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and/or time-consuming, but would be a routine undertaking for those of ordinary skill in the art having the benefit of this disclosure.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

“Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The term “for example” or “e.g.,” as used herein, is used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “operably linked” and operatively linked”, as used herein, refers to that union of the nucleic acid sequences that are linked in such a way, such that the coding regions are contiguous and in correct reading frame. Such sequences are typically contiguous, or substantially contiguous. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human.

As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

“Targeting moiety” is any factor that may facilitate targeting of a specific site by a particle. For example, the targeting moiety may be a chemical targeting moiety, a physical targeting moiety, a geometrical targeting moiety, or a combination thereof. The chemical targeting moiety may be a chemical group or molecule on a surface of the particle; the physical targeting moiety may be a specific physical property of the particle, such as a surface such or hydrophobicity; the geometrical targeting moiety includes a size and a shape of the particle. Further, the chemical targeting moiety may be a dendrimer, an antibody, an aptamer, which may be a thioaptamer, a ligand, an antibody, or a biomolecule that binds a particular receptor on the targeted site. A physical targeting moiety may be a surface charge. The charge may be introduced during the fabrication of the particle by using a chemical treatment such as a specific wash. For example, immersion of porous silica or oxidized silicon surface into water may lead to an acquisition of a negative charge on the surface. The surface charge may be also provided by an additional layer or by chemical chains, such as polymer chains, on the surface of the particle. For example, polyethylene glycol chains may be a source of a negative charge on the surface. Polyethylene glycol chains may be coated or covalently coupled to the surface using methods known to those of ordinary skill in the art.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

### EXAMPLES

The following example is included to demonstrate illustrative embodiments of the invention. It should be appreciated by those of ordinary skill in the art that the techniques disclosed in this example represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1—Aptamer-Engineered Natural Killer Cells for Cell-Specific Adaptive Immunotherapy

Natural Killer (NK) cells are a key component of the innate immune system as they can attack cancer cells without prior sensitization. However, due to lack of cell-specific receptors, NK cells are not innately able to perform targeted cancer immunotherapy. Aptamers are short single-stranded oligonucleotides that specifically recognize their targets with high affinity in a similar manner to antibodies. To render NK cells target-specificity, synthetic CD30-specific aptamers were anchored on cell surfaces to produce aptamer-engineered NK cells (ApEn-NK) without genetic alteration or cell damage. Under surface-anchored aptamer guidance, ApEn-NK specifically bound to CD30-expressing lymphoma cells, but did not react to off-target cells. The resulting specific cell binding of ApEn-NK triggered higher apoptosis/death rates of lymphoma cells compared to parental NK cells. Additionally, experiments with primary human NK cells demonstrated the potential of ApEn-NK to specifically target and kill lymphoma cells, thus presenting a potential new approach for targeted immunotherapy by NK cells.

Natural Killer (NK) cells are a unique subset of cytotoxic lymphocytes and a key component of the innate immune system[1,2]. In addition, the transfer safety of autologous, allogeneic, and cultured NK cells in adaptive cancer immunotherapy has been demonstrated[3-5]. In contrast to T-lymphocytes, NK cells do not express antigen-specific T cell receptors[6], but instead recognize target cells via an array of germ-line encoded surface ligands[7,8]. Therefore, NK cells are able to attack cancer cells without prior sensitization or clonal expansion[6,9]. NK cells can induce the death of targeted cancer cells through exocytosis of cytotoxic granules and can trigger apoptosis by activating cellular signaling pathways[10,11]. These unique, inherent properties confer high value to NK cells in adaptive cancer immunotherapy. However, due to their lack of cell-specific receptors, NK cells are not able to selectively target tumor cells, thus diminishing their immunotherapeutic potential. Chimeric antigen receptor (CAR)-T cell technology has emerged as a growing field and was recently FDA-approved for immunotherapy in certain cancer types[12,13]. Similarly, CAR-NK cells have also shown promising results for cancer immunotherapy[14-16]. However, the genetic manipulation used to generate CAR-T/NK cells may carry risks for patients, including transgene insertional mutagenesis[17,18]. Also, the production of CAR-NK cells is laborious and costly[19,20]. The development of a simpler and safer technology is necessary to overcome these technical and clinical challenges.

Aptamers are a group of short, single-stranded oligonucleotides with unique three-dimensional structures[21,22]. Similar to protein antibodies, aptamers specifically recognize their targets, which can include nucleic acids, proteins, cells, and even tissue, with high affinity[23-28]. Aptamers are considered “chemical antibodies,” given that they are chemically synthesized and have the ability to specifically bind their targets through three-dimensional recognition[29-31]. To target lymphoma in particular, CD30-specific aptamers have been previously developed and clinically validated [32,33].

In this Example, aptamer-engineered NK cells (ApEn-NK) were developed for targeted immunotherapy. To create cell-specificity, synthetic aptamers were anchored on the surface of NK cells via a simple biophysical reaction without genetic alteration. Under surface-anchored aptamer guidance, ApEn-NK specifically bound to lymphoma cells and subsequently triggered cell apoptosis and death. This exciting result opens a new and powerful avenue for cancer immunotherapy.

### Results

### Generation of Aptamer-Engineered NK Cells

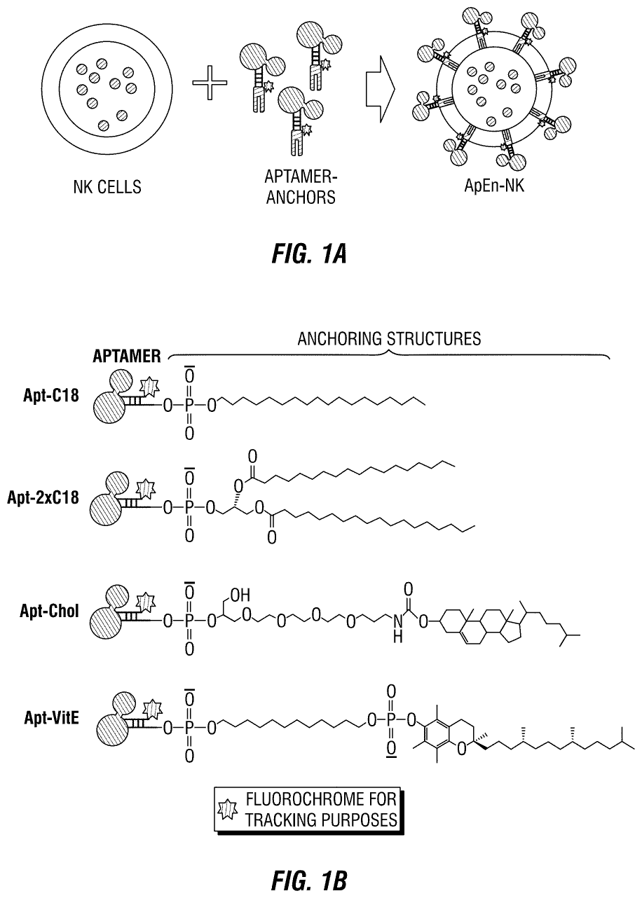

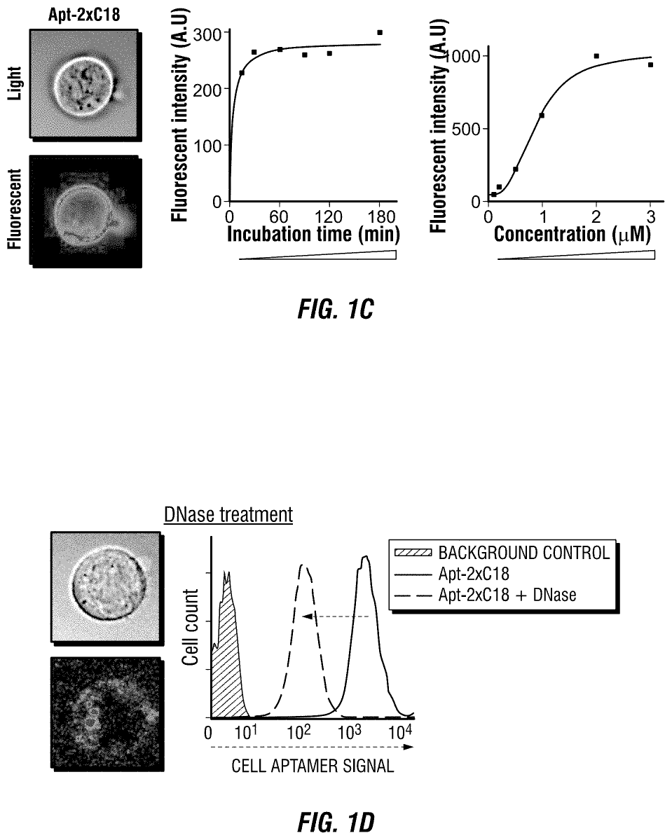

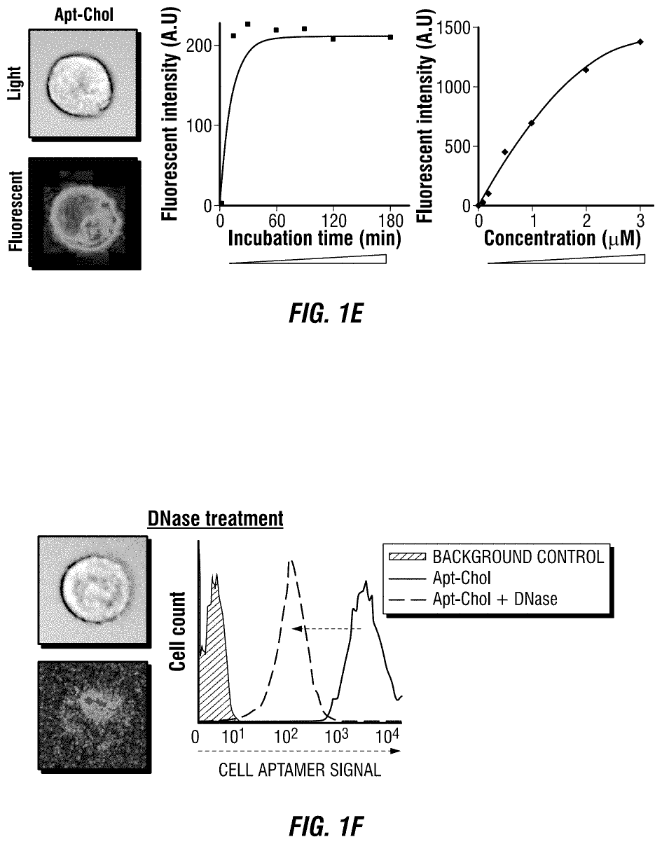

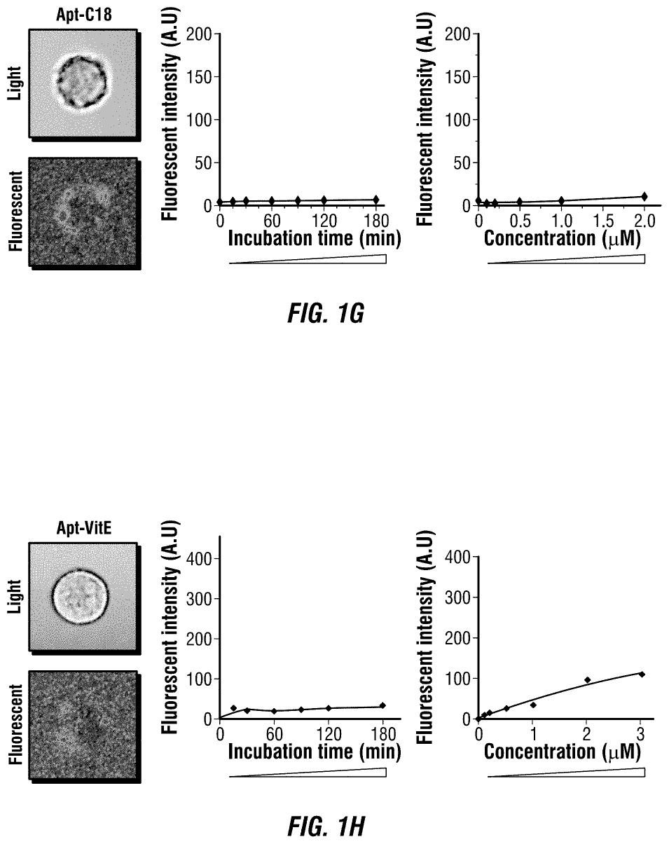

To render NK cells target-specificity, the ApEn-NK were generated by simply engineering synthetic aptamer-anchor structures on the cell surfaces via biophysical intercalation into the cell membrane (FIG. 1A). Lipophilic linkers have been widely used to anchor functional molecules on the membranes of living cells for different purposes, with no adverse impact on cell viability[34-37]. In this study, a CD30-specific ssDNA aptamer sequence [33] was synthesized to different lipophilic anchors, including single- or double-C18 hydrocarbon chains, cholesterol, or vitamin E, to produce the aptamer-anchor structures Apt-C18, Apt-2×C18, Apt-Chol, and Apt-VitE, respectively (FIG. 1B). For tracking purposes, fluorochrome Cy3 was attached to the 5′ end of the aptamer sequence. Because of their amphiphilic properties, the formed aptamer-anchor structures had the ability to precisely present hydrophilic aptamer sequences on the cell surface through intercalation of their hydrophobic anchors into the cell membrane.

To generate ApEn-NK, cultured NK92 cells were incubated with individual aptamer-anchoring structures under a physiological condition. Efficacy of cell surface-anchoring by synthetic Apt-2×C18, which contained two lipophilic chain anchor structures, was first studied. After a short incubation, confocal microscopy revealed fluorescent signals from the surface-anchored Apt-2×C18 on ApEn-NK with intact cell morphology (FIG. 1C). Quantitative analysis by flow cytometry demonstrated that the anchoring reaction rapidly achieved maximal levels in 30 min, and reached a plateau at a final concentration of 2 μM. For validation studies, the ApEn-NK were treated with DNase to digest the aptamers anchored on the cell surface. FIG. 1D shows that the DNase treatment resulted in a greater than 10-fold decrease in cell fluorescent signals derived from Apt-2×18C, thus confirming surface-anchoring of Apt-2×18C on ApEn-NK.

In addition, Apt-Chol, which contains a non-linear anchoring structure, was also used to produce ApEn-NK under the same reaction conditions. Confocal microscopy confirmed cell surface-anchoring of Apt-Chol as well as cytoplasmic penetration, likely due to cholesterol receptor-mediated endocytosis (FIG. 1E). Quantitative analysis demonstrated that the cell-anchoring reaction of Apt-Chol occurred rapidly and was dose-dependent. Similarly, exposure of the ApEn-NK to DNase treatment significantly reduced surface-anchored aptamer signals derived from Apt-Chol (FIG. 1F). In contrast, synthetic Apt-C18, which had single lipophilic chain anchor, failed to produce ApEn-NK (FIG. 1G), although it was able to be surface-anchored on cultured carcinoma cells under the same conditions (unpublished data). Moreover, Apt-VitE showed a minimal capacity to produce ApEn-NK, and an anchoring reaction plateau could not be reached even at a final concentration of up to 3 μM (FIG. 1H). Taken together, these findings indicate the potential of Apt-2×C18 and Apt-Chol structures to produce ApEn-NK.

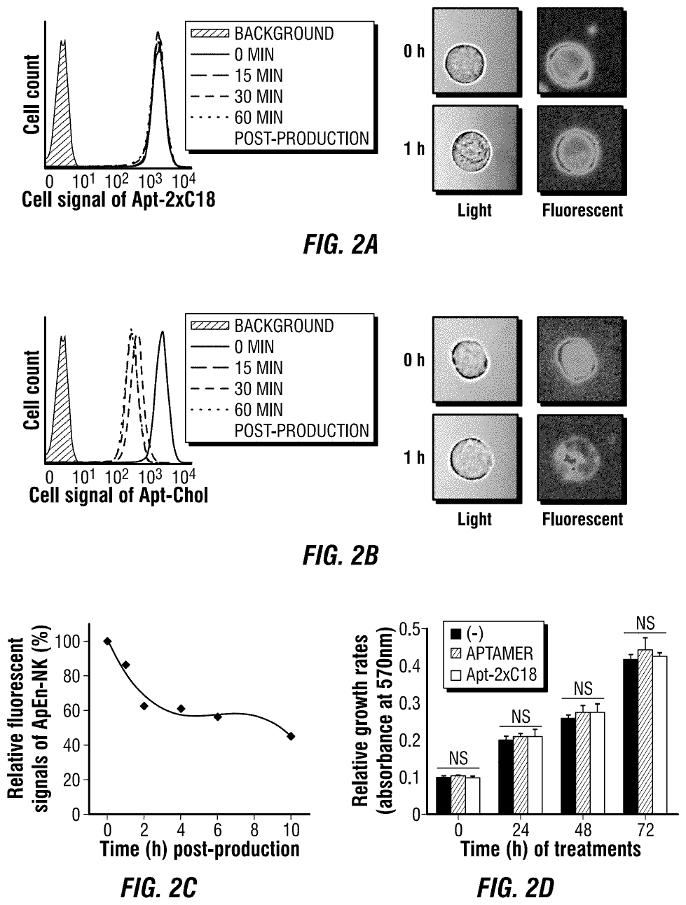

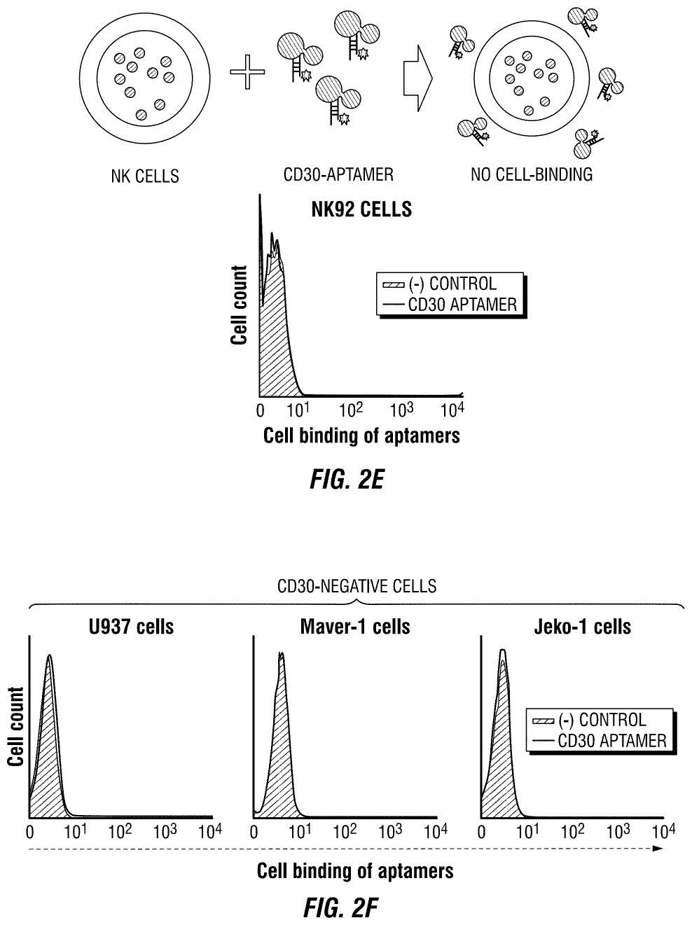

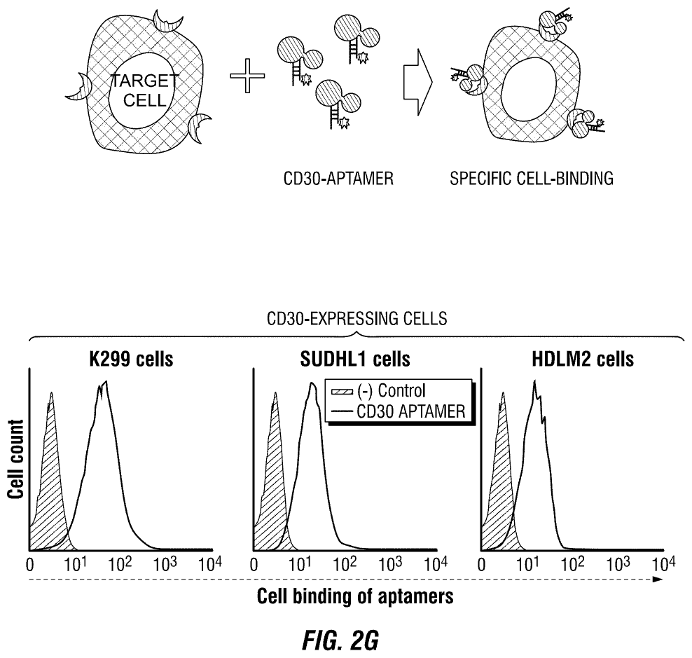

For biostability studies, the produced ApEn-NK were incubated in cell culture media, and changes in fluorescent signals of surface-anchored aptamers were monitored. Flow cytometry analysis revealed that ApEn-NK made with Apt-2×18C were stable, and a greater than 90% cellular signal of the anchored aptamers remained 60 min post-production (FIG. 2A and FIG. 2B). The remaining intact ApEn-NK carrying surface-anchored aptamers were also confirmed by confocal microscopy. In contrast, ApEn-NK made of Apt-Chol were not stable, as a greater than 80% reduction of the anchored aptamer signals was detected as early as 15 min post-production (FIG. 2C and FIG. 2D). Confocal microscopy also revealed nearly complete loss of the cell surface-anchored Apt-Chol signals in 60 min, despite the presence of scattered cytoplasmic signals. The observed instability limited Apt-Chol utility, and thus only ApEn-NK made of Apt-2×C18 were investigated in subsequent studies.

For extended biostability validation, the ApEn-NK were incubated in cell culture media over a time course. Quantitative flow cytometry revealed that more than 40% of cell signals of anchored aptamer remained on ApEn-NK 10 hr post-production (FIG. 2E). To evaluate the biocompatibility of synthetic aptamer-anchor structures, cultured NK92 cells were continuously exposed to Apt-2×C18 for three days while changes in cell growth rates were monitored. FIG. 2F shows that the presence of Apt-2×C18 in cultures had no adverse effects on NK92 cell growth, as compared to non-treatment or aptamer sequences with control cells.

Finally, to confirm that NK cell-anchoring of Apt-2×18C was through anchor structures, NK92 cells were incubated with only Cy3-labeled aptamer sequences (FIG. 2G). Flow cytometry analysis showed that sole aptamer sequences could neither be anchored on NK92 cells nor bind to NK92 cells that lack CD30 expression. To rule out non-specific aptamer-cell interaction, additional CD30-negative cells were also tested, and no cell binding of aptamer sequences was detected by flow cytometry analysis (FIG. 2I). To confirm targeting specificity, CD30-positive lymphoma cells were treated with aptamer sequences [33] and the resulting specific cell-binding by aptamers was determined by flow cytometry (FIG. 2J and FIG. K). These findings indicate that cell surface-anchoring of Apt-2×18C was mediated by its anchor structures.

### Specific Binding of ApEn-NK to Lymphoma Cells

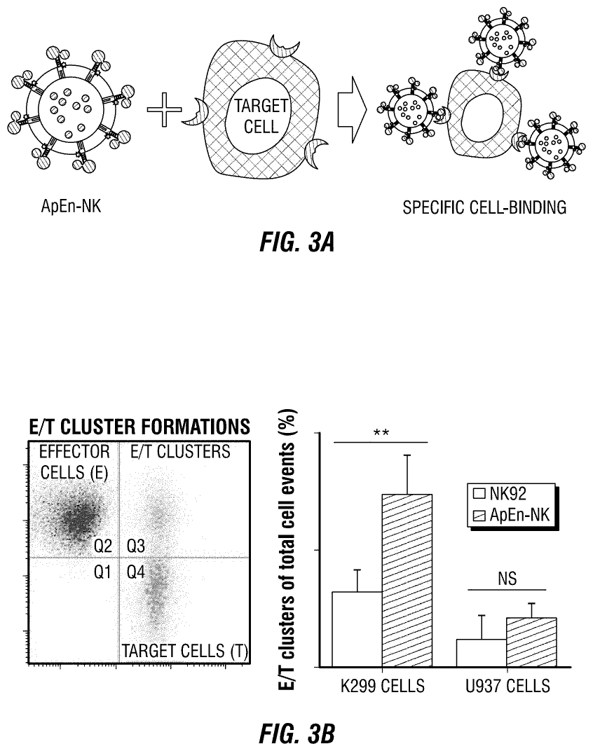

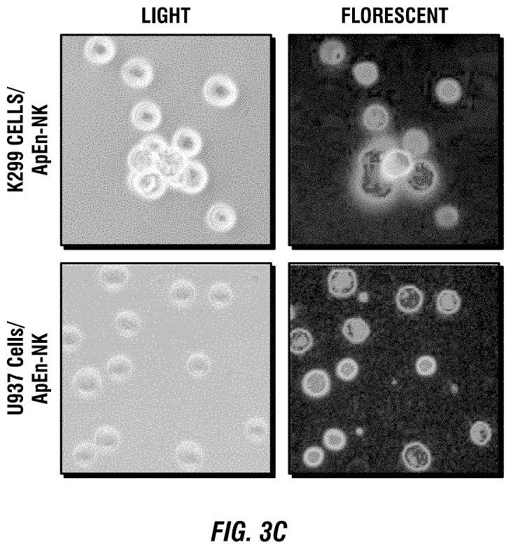

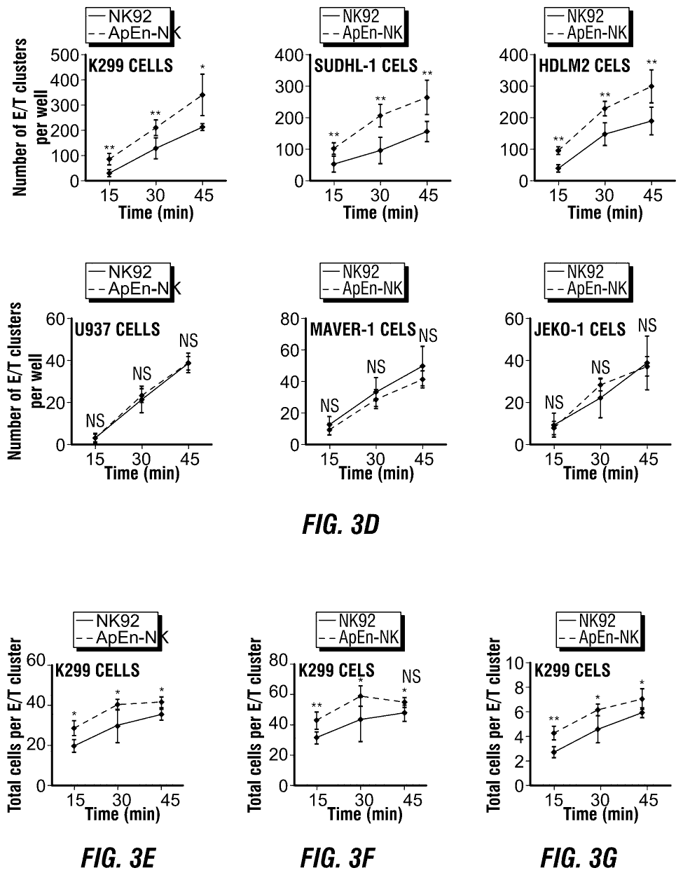

It is expected that under the guidance of surface-anchored aptamers, ApEn-NK would be able to specifically target lymphoma cells as illustrated in FIG. 3A. To test this hypothesis using cell binding assays, CD30-expressing K299 lymphoma cells were pre-stained with Calcein-AM (green fluorescence), and ApEn-NK cells were tracked by the red fluorescence of surface-anchored Apt-2×C18. Equal amounts of ApEn-NK (Effector cells) and lymphoma cells (Target cells) were mixed. In control experiments, parental NK92 cells were pre-stained with Red-Orange AM and used to replace ApEn-NK in the cell mixture. Resultant cell binding was analyzed by flow cytometry 30 min post-incubation. As shown in FIG. 3A and FIG. 3B, different cell populations were separated and gated, specifically, including Effector cells (E) in red, Target cells (T) in green, and E/T clusters containing both red and green signals [38]. Quantitative analysis revealed that ApEn-NK specifically targeted lymphoma cells and formed E/T clusters significantly more than that observed in control experiments containing NK92 cells (12.38% vs. 8.22% mean cluster formation rates, p<0.05). Notably, in mixtures of CD30-negative U937 cells, similar E/T cluster formation baselines were detected with ApEn-NK or NK92 cells (7.11% vs. 6.2% mean cluster formation rates).

For further characterization, cell mixtures were examined under a fluorescence microscope, confirming E/T cluster formation (FIG. 3D). In addition, time course studies with CD30-positive lymphoma cells (K299, SUDHL-1, and HDLM2 cell lines) also revealed that ApEn-NK induced significantly higher numbers of E/T clusters per well of culture plates than that by parental NK92 cells (FIG. 3E and FIG. 3F). However, ApEn-NK and NK92 cells had very similar E/T cluster formation rates in mixtures containing CD30-negative cells (U937, Maver-1, and Jeko-1 cell lines), indicating that ApEn-NK were target cell-specific. Moreover, cell numbers of each E/T cluster were manually counted under a fluorescence microscope. In comparison to parental NK92 cells, ApEn-NK induced larger E/T clusters that contained more total cells (FIG. 3G) and higher numbers of both ApEn-NK and target lymphoma cells (FIG. 3G). Taken together, these findings demonstrated the specific binding capacity of ApEn-NK to target lymphoma cells.

### Specific Killing of Lymphoma Cells by ApEn-NK

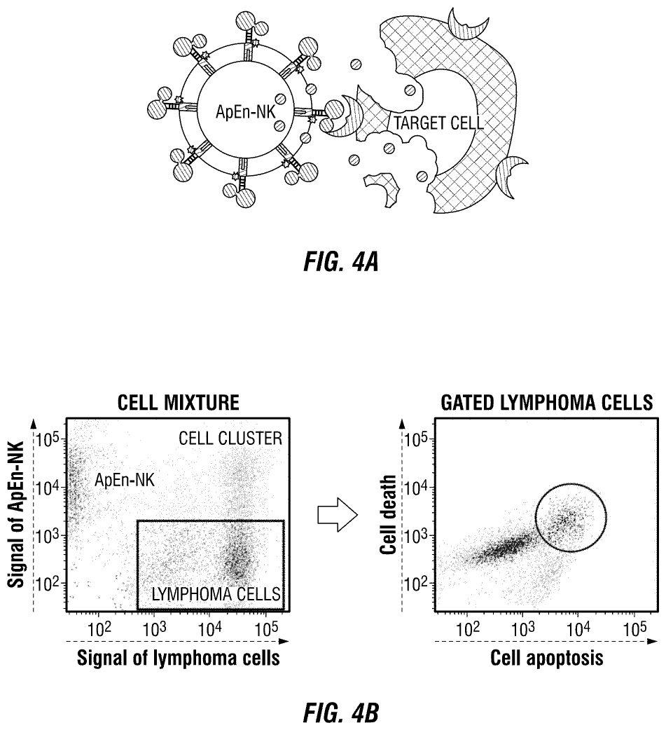

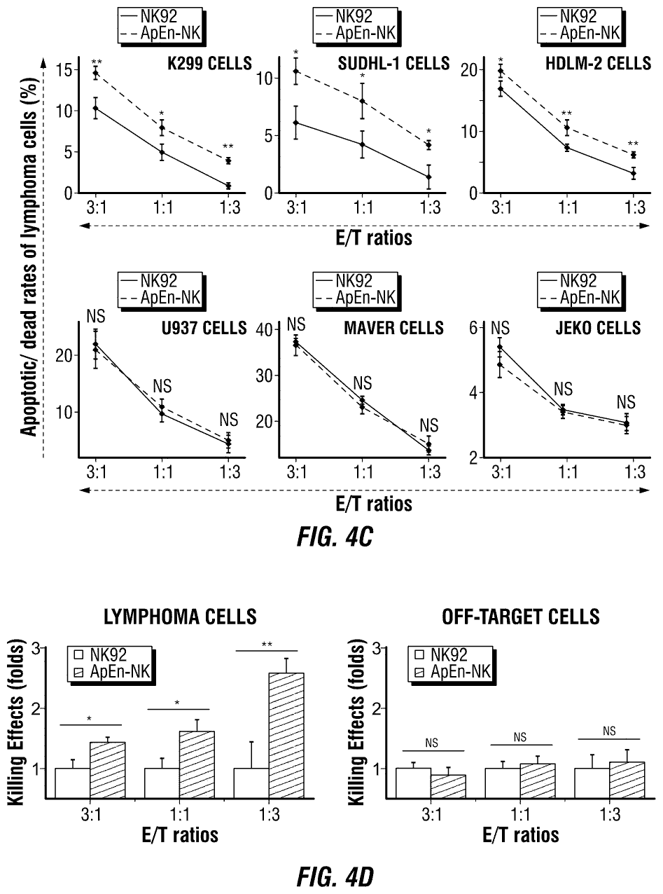

Given that NK cells are able to attack cancer cells without prior sensitization, acquired specific cell-binding capacity should result in higher killing efficacy of ApEn-NK (FIG. 4A). For cell functional assays, ApEn-NK were incubated with lymphoma cells at E/T ratios of 3:1, 1:1, or 1:3, and the resultant changes in apoptosis and death rates of target cells were evaluated. For identification purposes, lymphoma cells were pre-stained with green fluorescence and ApEn-NK cells were tracked by red fluorescence of anchored aptamers, as described above. After incubation, cells were harvested and stained with Cy5 Annexin V and eFluor 450 dyes to mark apoptotic and dead cells, respectively. To evaluate killing efficacy, individual cell populations were first separated and gated by flow cytometry, including lymphoma cells, ApEn-NK, and formed cell clusters (FIG. 4B and FIG. 4E). Subsequently, the gated lymphoma cells were further analyzed to determine changes in cell apoptosis/death (FIG. 4E).

In comparison to parental NK92 cells, ApEn-NK induced significant increases in apoptosis/death of target lymphoma cells (K299, SUDHL-1, and HDLM-2 cells lines that express CD30) at all tested E/T ratios (FIG. 4C). In contrast, no enhanced killing effects on off-target control cells (U937, Maver-1, and Jeko-1 cell lines) were observed, demonstrating that the ApEn-NK killing effect was target cell-specific. Interestingly, statistical analysis of all tested lymphoma cells revealed the highest enhancement of killing effects at the lowest E/T ratio. In comparison to non-specific background rates determined by parental NK92 cells, 44%, 62%, and 168% increases in lymphoma cell killing at E/T ratios 3:1, 1:1, and 1:3, respectively, were detected (FIG. 4D).

Characterization of ApEn-NK from Primary Human NK Cells

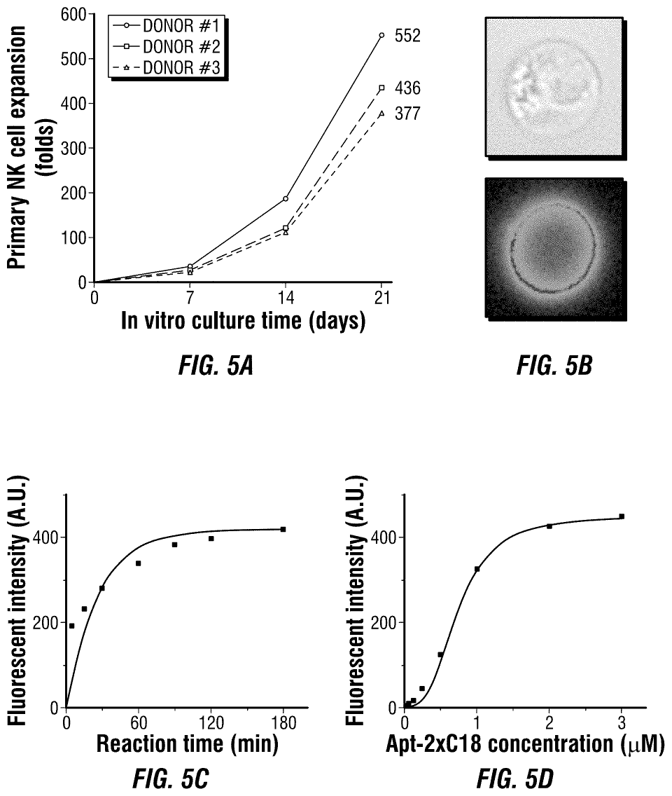

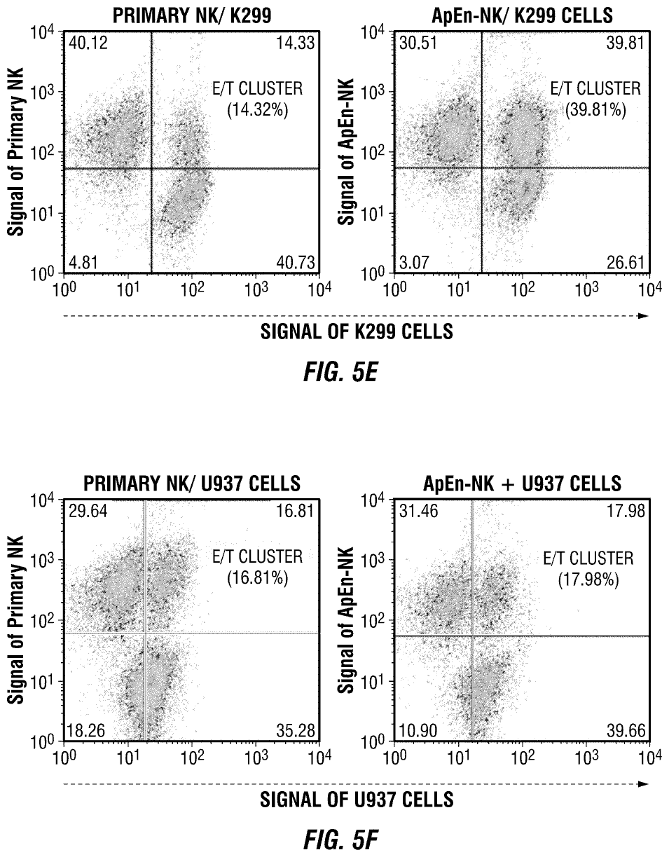

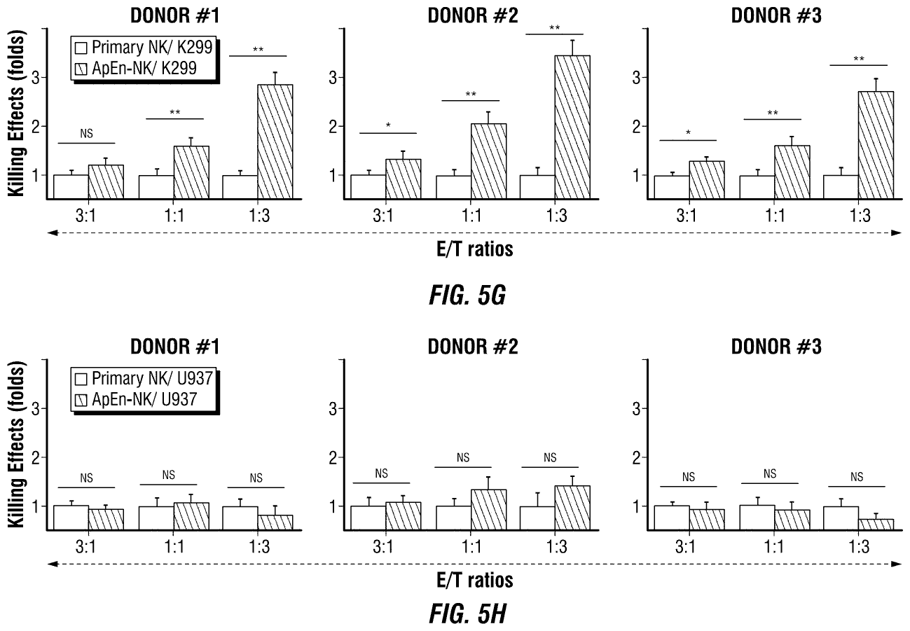

To determine if these results would replicate in human models, primary NK cells were isolated from three healthy human donors and cultured for in vitro expansion. After culturing for three weeks, primary NK cells from individual donors were expanded up to 377, 436, and 552 folds, respective to each donor (FIG. 5A). Subsequently, ApEn-NK were produced in these cells with Apt-2×C18 aptamers, and successful surface-anchoring of aptamers on ApEn-NK was confirmed by confocal microscopy, as previously described (FIG. 5B). In addition, changes in fluorescent signals of ApEn-NK derived from surface-anchored aptamers were dynamically monitored by flow cytometry analysis. Time course assays demonstrated that the aptamer-anchoring reaction on primary NK cells occurred rapidly, and achieved a maximal level within 90 min (FIG. 5C). Moreover, dose course studies showed that the aptamer-anchoring reaction was dose-dependent, and reached a plateau at 2 μM final concentration of Apt-2×C18 structures (FIG. 5D).

For cell binding assays, target cells (K299 and U937) were pre-stained with Calcein-AM, and ApEn-NK cells were tracked by fluorescent signal of surface-anchored aptamers. In control experiments, primary human NK cells were pre-stained with Red-Orange AM and used instead of ApEn-NK. ApEn-NK or primary NK cells were incubated with target cells and resultant E/T clusters were then quantified by flow cytometry, as previously described. In comparison to the non-specific baseline of cell binding established by primary NK cells, ApEn-NK induced significantly more E/T clusters with CD30-expressing K299 cells (14.32% vs. 39.81%, p<0.5) (FIG. 5E). However, similar E/T cluster formation with CD30-negative U937 cells were observed in cell mixtures containing primary NK cells or ApEn-NK under the same conditions (FIG. 5F). These findings indicated that ApEn-NK derived from primary NK cells were able to specifically bind lymphoma cells and did not react to off-target cells.

Finally, to evaluate immunotherapeutic potential, target cells (K299 or U937) were treated with ApEn-NK derived from the three healthy donors at the previously defined E/T ratios. In control experiments, parental primary NK cells derived from the same donor were used to replace ApEn-NK. The resultant apoptosis/death rates of target cells were then quantified by flow cytometry analysis, as previously described. FIG. 5G shows that in comparison to paired parental NK cells, ApEn-NK treatment induced a significant increase in apoptosis/death of CD30-expressing K299 lymphoma cells. Notably, although enhanced killing effects were observed in all tested E/T ratios, the highest increased killing effect was observed at the lowest E/T ratio. Quantitative analysis of the killing effects of three ApEn-NK lines revealed a 28% increase (mean±6%) at E/T ratio 3:1, a 76% increase at E/T ratio 1:1 (mean±26%), and a 201% increase at E/T ratio 1:3 (mean±39%) from the baseline effects. In contrast, no enhanced killing effect of ApEn-NK on CD30-negative U937 cells was observed under the same conditions (FIG. 5H). Taken together, these findings demonstrate the immunotherapeutic potential of ApEn-NK to specifically attack lymphoma cells.

### DISCUSSION

This study validates a unique approach to render NK cells target-specificity by simply anchoring oligonucleotide aptamers on the cell surface without genetic alteration or cell damage. Under the guidance of surface-anchored aptamers, ApEn-NK cells specifically targeted and subsequently induced apoptosis/death of targeted lymphoma cells. This ApEn-NK platform has several unique features as compared to CAR-T/NK technology[19, 20, 39]. First, generation of ApEn-NK cells is a simple, time- and labor-efficient process as it involves a single-step engineering reaction and can be completed in 30 min. Notably, the engineering reaction was highly effective, and all NK cells were surface-anchored with amphiphilic aptamer-anchor structures without alteration of cellular properties. The simplicity and efficiency of the engineering process render ApEn-NK cells clinically valuable for rapid personalized adaptive immunotherapy. Secondly, because they are composed of natural nucleic acids, aptamer-anchor structures are biocompatible and biodegradable, and will be risk-free for clinical use. Importantly, manufacturing of ApEn-NK cells does not involve genetic manipulation and/or viral gene transfection, which are required for CAR-T/NK generation[2, 18, 40] and can carry undefined risks for patients[41-43]. Finally, in vitro studies demonstrated that ApEn-NK were able to efficiently kill target lymphoma cells, with the highest enhancement in the lowest tested E/T ratio 1:3 (FIG. 4D). These findings strongly suggest the suitability of ApEn-NK for immunotherapy as the in vivo E/T ratios at tumor sites are expected to be significantly lower than those in in vitro experiments.

Phospholipids are major components of cell membranes, constituting the membrane's bilayer structure. Cholesterol is an essential component of the membrane and is dispersed between phospholipids to maintain membrane integrity and flexibility [44]. Notably, phospholipid and cholesterol derivatives have the capacity to intercalate between cell membranes via hydrophobic interactions, and thus have been used as linkers for fluorescence labeling of membranes [36, 37, 45]. In this study, different phospholipid and cholesterol derivatives were tested in the production of amphiphilic aptamer-anchor structures (FIG. 1B). The single hydrocarbon chain anchor failed to engineer aptamers on NK cells, although this strategy is widely used for linkers carrying single fluorochrome molecules. Interestingly, Apt-2×C18 achieved rapid and stable surface-anchoring of aptamers on NK cells. These findings suggest that an optimal balance between the hydrophobic and hydrophilic properties of amphiphilic aptamer-anchor structures is critical for successful membrane intercalation. Cholesterols instead of phospholipids were also studied in the generation of aptamer-anchor structures. Apt-Chol had high efficacy in the surface-anchoring of aptamers on NK cells (FIG. 1E), but poor stability on cell membranes, which is likely due to receptor-mediated internalization (FIG. 2C and FIG. 2D). In addition, vitamin E, an essential component of membrane lipids[46], was tested as an anchor; however, Apt-VitE showed minimal capacity for surface-anchoring on NK cells. Moreover, our unpublished results showed that the surface-anchoring capacity of different linkers varied significantly among engineered cells. These findings demonstrated that precision optimization of aptamer-anchor structures is essential to achieve maximum efficacy of cell surface-anchoring. Thus, it is necessary to carefully validate the aptamer-anchor structures under each set of clinical conditions.

Under surface-anchored aptamer guidance, the ApEn-NK specifically targeted lymphoma cells, leading to the formation of stable E/T clusters. Notably, the surface-anchored aptamers enhanced cell-specific binding, but had no effect on NK cell activity because of the lack of intracellular signaling domains. Therefore, it is reasonable to conclude that the observed immunotherapeutic effects on targeted lymphoma cells resulted from aptamer-mediated specific cell binding and the innate killing capacity of ApEn-NK. To improve immunotherapeutic potential, ApEn-NK cells could be stimulated and activated in vitro prior to adoptive transfer. Theoretically, the aptamer-anchoring system can be combined with a cell activation approach, creating ApEn-NK specific cell-binding and enhancing targeted cell killing for improved immunotherapy. It would be of interest to compare the immunotherapeutic efficacy of ApEn-NK derived from autologous- and allogeneic-donors because autologous NK cells will have no risk for the patients.

Biostability studies revealed that the surface-anchored aptamers were stable up to 10 h post-ApEn-NK production (FIG. 2E), with only a moderate decrease likely due to nuclease degradation and/or dilution as NK cells divided. To enhance in vivo biostability, nuclease-resistant aptamer sequences can be synthesized through chemical modifications. Although the minimal number of surface-anchored aptamers required for cell targeting is unknown, the use of high-affinity aptamer sequences can improve the cell-binding capacity and functional lifetime of ApEn-NK upon dividing in vivo. Finally, our preclinical studies demonstrated the possibility of producing ApEn-NK using primary human NK cells derived from healthy donors for specific killing of target lymphoma cells.

### Methods

Reagents and cells: Cell lines Karpas 299 (K299, T-cell lymphoma), SUDHL-1 (diffuse histiocytic lymphoma), HDLM2, (Hodgkin lymphoma), U937 (histiocytic lymphoma), Jeko-1 (B-cell lymphoma), and Maver-1 (mantle cell lymphoma) were purchased from American Type Culture Collection (ATCC, Manassas, Va., USA) and maintained in RPMI 1640 medium (Thermo Fisher Scientific, Rockford, Ill., USA) containing 10% FBS and 100 IU/mL penicillin-streptomycin. NK92 cells (NK cell lymphoma) from ATCC were cultured in alpha minimum essential medium (αMEM) (Gibco, Grand Island, N.Y., USA) supplemented with 12.5% Fetal Bovine Serum (FBS) (Atlanta Premium, Atlanta, Ga., USA), 12.5% horse serum from ATCC, 0.2 mmol/L inositol (Sigma Aldrich, St. Louis, Mo., USA), 0.1 mmol/L β-mercaptoethanol (Sigma Aldrich), 0.02 mmol/L folic acid (Sigma Aldrich), 200 U/mL recombinant IL-2 (Peprotech, Rocky Hill, N.J., USA), and 100 IU/mL penicillin-streptomycin (Corning, Corning, N.Y., USA).

To isolate primary human NK cells, peripheral blood from anonymized healthy donors was used with an IRB-approved protocol. Mononuclear cells in blood buffy coats were isolated by a density-gradient technique (Ficoll-Histopaque; Sigma, St Louis, Mo., USA) and CD56+NK cells were then purified using an NK isolate kit (Miltenyi Biotec, San Diego, Calif., USA). For cell expansion, primary NK cells were cultured in RPMI 1640 medium (Corning, N.Y., USA) in the presence of irradiated (100 Gy) feeder cells of K562-mb15-41 BBL obtained from St. Jude's Children's Research Hospital at 2:1 ratio of feeder:NK cells, and supplemented with 5% human AB serum (Sigma), recombinant human IL2 (200 IU/mL, PeproTech, Rocky Hill, N.J., USA), recombinant human IL15 (10 ng/mL, PeproTech), and recombinant human IL21 (1 ng/mL, PeproTech). Cultures were expanded every other day with fresh media and irradiated feeder cells were replaced every week. NK cell expansion was monitored by cell counting on day 7, 14, and 21.

Cell-binding assay of aptamer sequences: The CD30-specific ssDNA aptamer sequence, 5′-ACTGGGCGAAACAAGTCTATTGACTATGAGC-3′ (SEQ ID NO:1), was labeled with fluorochrome Cy3 at the 5′end for tracking purposes, as previously reported[33]. Cultured cells (5×10⁵), including the human NK cell line (NK92), CD30-expressing lymphoma cell lines (K299, SUDHL-1, and HDLM2), and CD30-negative lymphoma/leukemia cell lines (U937, Jeko-1, and Maver-1) were incubated with 200 nM aptamer probes at room temperature (RT) for 30 min in Dulbecco's phosphate-buffered saline (DPBS) (GE Healthcare, Chicago, Ill., USA). Cells were washed once with DPBS and resulting cell binding of aptamer probes was quantified by flow cytometry (LSRII, BD Biosciences, San Jose, Calif., USA). The same set of cells treated with random ssDNA sequences of the same length were set as negative/baseline controls for the cell-binding assay.

Design of aptamer-anchor structures: To identify optimal aptamer-anchor structures for engineering of ApEn-NK, the aptamer sequences were conjugated at the 3′ end to different lipophilic anchor molecules, including: single and double C18 hydrocarbon chains, cholesterol (via a 15-atom triethylene glycol spacer), or vitamin E (via a 12-carbon spacer), as shown in FIG. 1B. For tracking purposes, fluorochrome Cy3 was conjugated at the 5′ end as a reporter. All aptamer-anchor structures were synthesized and purified by Integrated DNA Technologies (IDT, Coralville, Iowa, USA), and stored in nuclease-free water at −20° C. until used.

Production and characterization of ApEn-NK: Cultured NK92 cells or fresh primary human NK cells (5×10⁵) were incubated with individual aptamer-anchor structures in DPBS at RT with intermittent mixing to produce ApEn-NK cells. The concentrations of aptamer-anchor structures and reaction times are described in the individual experiments. After the anchoring reaction, the produced ApEn-NK were washed once and resuspended in DPBS. Both dose and time course studies were conducted to determine the optimal reaction conditions and optimal concentration of aptamer-anchor structures for ApEn-NK production.

Surface-anchoring of aptamer sequences on ApEn-NK was confirmed with a confocal microscope (FluoView™ FV1000, Olympus America, Melville, N.Y., USA) at ×60 magnification on bright field and the Cy3 channel. In addition, cellular fluorescence signals derived from surface-anchored aptamers on ApEn-NK were quantified by flow cytometry analysis. Intact parental NK92 or primary human NK cells were used for assay baseline controls. To evaluate biostability, ApEn-NK cells were maintained in culture media, and residual cellular signals of surface-anchored aptamers were quantified at different time points, as described in the experiments.

To validate the presence of aptamer sequences on the exterior of the cell membranes, ApEn-NK were treated with 250 U/mL DNAse (Sigma Aldrich) in DPBS at 37° C. for 30 min. ApEn-NK cells were washed with DPBS once, and residual cellular signals of surface-anchored aptamers were quantified by flow cytometry.

Cell proliferation assay: To rule out cytotoxicity, NK92 cells (5×10⁴) were incubated with aptamer-anchor structures Apt-2×C18 under the same conditions to produce ApEn-NK or equimolar amounts of CD30 aptamer sequences alone in control experiments. Cells were then seeded into complete culture media, and cultured in 96-well plates. Changes in cell proliferation were monitored via MTT (3-[4,5-dimethylthiazol-2-yl]-2,5-diphenyltetrazolium bromide) assay at 24, 48, and 72 h. The absorbance in each well at 570 nm was measured with a microplate reader (GE Healthcare).

Quantification of cell cluster formation: For identification purpose, lymphoma cells (Target cells, T) were pre-stained with 100 nmol/L Calcein-AM (green fluorescence), and ApEn-NK (Effector cells, E) were tracked by red fluorescence signals from the surface-anchored aptamers. In control experiments, parental NK cells were pre-stained with 100 nmol/L Calcein Red-Orange AM (Life Technologies, Carlsbad, Calif., USA) to replace AnEn-NK. For the cell binding reaction, equal amounts of effector and target cells (1×10⁵) were mixed at different ratios in 500 μL RPMI 1640 containing 10% FBS. Subsequently, the mixture was incubated in a 5 mL polystyrene round-bottom tube with constant gentle shaking on a shaking-bed (GE Healthcare) at RT for 30 min. Constant shaking mimics in vivo dynamic blood flow and also prevents non-specific cell clotting due to gravity-induced precipitation. Cell mixtures were analyzed by flow cytometry, and the formed E/T clusters were quantified. Given that E/T clusters emitted fluorescence signals with both effector and target cells, they were selectively gated, and calculated as the percentage of total detected cell events[38]. Each set of samples was analyzed in triplicate, and the analysis was repeated ≥3 times.

For the time course study, equal amounts of effector and target cells (2×10⁴) were mixed in 2 mL RPMI 1640 containing 10% FBS. Cell mixtures were incubated in a 12-well plate with constant gentle shaking for 15, 30, or 45 min. Individual wells were then examined under a fluorescence microscope to count the number of formed E/T clusters that were aggregates of more than 4 cells and contained both effector (red signal) and target (green signal) cells. In addition, the number of total, effector, and target cells per formed E/T cluster were manually counted, and a mean value with ±SD was calculated from 8 or more randomly observed clusters.

Cell killing assays: ApEn-NK were produced by incubating 5×10⁵ cultured NK92 cells with 1 μmol/L Apt-2×C18 in DPBS at RT for 30 min. For tracking purposes, lymphoma (K299, HDLM2, and SU-DHL-1 cell lines) or control cells (U937, Jeko-1, and Maver-1 cell lines) were labeled with 1 μmol/L Calcein-AM. ApEn-NK cells were mixed with target cells in 2 mL RPMI 1640 containing 10% FBS in 12-well plates at E:T ratios of 3:1, 1:1, and 1:3. Notably, all cell mixtures contained equal amounts of target cells (3×10⁴) with variable numbers of ApEn-NK cells to achieve different E:T ratios as noted in the figure. Cell mixtures were gently shaken on a shaking bed at RT for 30 min to avoid non-specific cell binding, then incubated in a humidified atmosphere with 5% CO₂ at 37° C. for 4 h. Finally, cell mixtures were labeled with Cy5 Annexin V (BD Bioscience, Waltham, Mass., USA) and Dye eFluor 450 (eBioscience, Waltham, Mass., USA) for 30 min, and analyzed by flow cytometry. First, different cell populations were gated, including ApEn-NK bearing the red fluorescent signals from the surface-anchored aptamers, lymphoma cells pre-stained with green fluorescence Calcein-AM, and E/T clusters that emitted both red and green signals. Subsequently, the gated lymphoma cell population was further analyzed for cell apoptotic and death rates based on cellular signals of Cy5 Annexin V and eFluor 450, respectively. The resulting apoptosis/death rates over the total gated lymphoma cells (%) with ±SD were calculated. Each set of samples was analyzed in triplicate and the same experiments were repeated ≥3 times with similar findings.

For primary NK cell killing assay, expanded primary NK cells were incubated with 1 μmol/L Cy3-Apt-2×C18 in DPBS at RT for 30 min. Target cells K299 and U937 were labeled with 100 nmol/L Calcein-AM in DPBS at RT for 30 min. After washing twice, NK cells and target cells were mixed in RPMI1640 supplemented with 10% FBS and and 500 IU/ml recombinant human IL2 in V-bottom 96-well plate. Plate was centrifuged for 1 min at 100 g to initiate cell contact and incubated at at 37° C. for 4 hr Cells were mixed by pipetting with a 100 μL pipetter in order to uniformly suspend the released calcein followed with spinning down plate at 100 g for 5 minutes to pellet the cells, and then 100 μL of the supernatant was transferred to a new plate. Plate was read using a fluorescent plate reader (excitation filter 485 nm, emission filter 530 nm) and the percent specific lysis was calculated according to the formula [(test release-spontaneous release)/(maximum release−spontaneous release)]×100. Each set of samples was analyzed in triplicate and the same experiments were repeated ≥3 times with similar findings.

Statistical analysis: The data were presented as the mean±standard deviation (SD). Sample size (n) for each statistical analysis was a minimum of three. Statistical analysis and graph plotting were performed with OriginPro 8.5. Differences between groups were determined by one-way Analysis of variance (ANOVA). p values of *<0.05 and **<0.01 were considered statistically significant. All the experiments have been repeated for at least three times with similar findings.

### REFERENCES

The following references, to the extent that they provide exemplary procedural or other details supplementary to those set forth herein, are specifically incorporated herein in their entirety by express reference thereto:
1. Zhao, E., Xu, H., Wang, L., Kryczek, I., Wu, K., Hu, Y., Wang, G., Zou, W., Cellular & molecular immunology 2012, 9 (1), 11-19.
2. Rezvani, K., Rouce, R., Liu, E., Shpall, E., Molecular Therapy 2017.
3. Rubnitz, J. E., Inaba, H., Ribeiro, R. C., Pounds, S., Rooney, B., Bell, T., Pui, C.-H., Leung, W., Journal of clinical oncology 2010, 28 (6), 955-959.
4. Miller, J. S., Soignier, Y., Panoskaltsis-Mortari, A., McNearney, S. A., Yun, G. H., Fautsch, S. K., McKenna, D., Le, C., Defor, T. E., Burns, L. J., Blood 2005, 105 (8), 3051-3057.
5. Olson, J. A., Leveson-Gower, D. B., Gill, S., Baker, J., Beilhack, A., Negrin, R. S., Blood 2010, 115 (21), 4293-4301.
6. Davies, J. O., Stringaris, K., Barrett, A. J., Rezvani, K., Cytotherapy 2014, 16 (11), 1453-1466.
7. Dahlberg, C. I., Sarhan, D., Chrobok, M., Duru, A. D., Alici, E., Frontiers in immunology 2015, 6.
8. Venstrom, J. M., Pittari, G., Gooley, T. A., Chewning, J. H., Spellman, S., Haagenson, M., Gallagher, M. M., Malkki, M., Petersdorf, E., Dupont, B., New England Journal of Medicine 2012, 367 (9), 805-816.
9. Childs, R. W., Carlsten, M., Nature reviews Drug discovery 2015, 14 (7), 487-498.
10. Fang, F., Xiao, W., Tian, Z., Semin Immunol 2017, 31, 37-54. DOI 10.1016/j.smim.2017.07.009.
11. Lowin, B., Peitsch, M., Tschopp, J., Perforin and granzymes: crucial effector molecules in cytolytic T lymphocyte and natural killer cell-mediated cytotoxicity. In Pathways for Cytolysis, Springer: 1995; pp 1-24.
12. Maus, M. V., Grupp, S. A., Porter, D. L., June, C. H., Blood 2014, 123 (17), 2625-2635.
13. Grupp, S. A., Kalos, M., Barrett, D., Aplenc, R., Porter, D. L., Rheingold, S. R., Teachey, D. T., Chew, A., Hauck, B., Wright, J. F., New England Journal of Medicine 2013, 368 (16), 1509-1518.
14. Chu, J., Deng, Y., Benson Jr, D. M., He, S., Hughes, T., Zhang, J., Peng, Y., Mao, H., Yi, L., Ghoshal, K., Leukemia 2014, 28 (4), 917.
15. Oelsner, S., Friede, M. E., Zhang, C., Wagner, J., Badura, S., Bader, P., Ullrich, E., Ottmann, O. G., Klingemann, H., Tonn, T., Wels, W. S., Cytotherapy 2017, 19 (2), 235-249. DOI 10.1016/j.jcyt.2016.10.009.
16. Tang, X., Yang, L., Li, Z., Nalin, A. P., Dai, H., Xu, T., Yin, J., You, F., Zhu, M., Shen, W., Chen, G., Zhu, X., Wu, D., Yu, J., Am J Cancer Res 2018, 8 (6), 1083-1089.
17. Glienke, W., Esser, R., Priesner, C., Suerth, J. D., Schambach, A., Wels, W. S., Grez, M., Kloess, S., Arseniev, L., Koehl, U., Frontiers in pharmacology 2015, 6.
18. Ren, J., Zhao, Y., 2017, 8 (9), 634-643. DOI 10.1007/s13238-017-0410-x.
19. Li, L., Liu, L. N., Feller, S., Allen, C., Shivakumar, R., Fratantoni, J., Wolfraim, L. A., Fujisaki, H., Campana, D., Chopas, N., Cancer gene therapy 2010, 17 (3), 147.
20. Piscopo, N. J., Mueller, K. P., Das, A., Hematti, P., Murphy, W. L., Palecek, S. P., Capitini, C. M., Saha, K., Biotechnol J 2018, 13 (2). DOI 10.1002/biot.201700095.
21. Xiang, D., Shigdar, S., Qiao, G., Wang, T., Kouzani, A. Z., Zhou, S. F., Kong, L., Li, Y., Pu, C., Duan, W., Theranostics 2015, 5 (1), 23-42. DOI 10.7150/thno.10202.
22. Nimjee, S. M., White, R. R., Becker, R. C., Sullenger, B. A., Annual review of pharmacology and toxicology 2017, 57, 61-79. DOI 10.1146/annurev-pharmtox-010716-104558.
23. Sun, H., Zhu, X., Lu, P. Y., Rosato, R. R., Tan, W., Zu, Y., Molecular Therapy—Nucleic Acids 2014, 3 (8), e182.
24. Watrin, M., Dausse, E., Lebars, I., Rayner, B., Bugaut, A., Toulme, J. J., Methods in molecular biology (Clifton, N.J.) 2009, 535, 79-105. DOI 10.1007/978-1-59745-557-2_6.
25. McGivney, J. B. t., Csordas, A. T., Walker, F. M., Bagley, E. R., Gruber, E. M., Mage, P. L., Casas-Finet, J., Nakamoto, M. A., Eisenstein, M., Larkin, C. J., Strouse, R. J., Soh, H. T., 2018, 90 (5), 3262-3269. DOI 10.1021/acs.analchem.7b04775.
26. Kanlikilicer, P., Ozpolat, B., Aslan, B., Bayraktar, R., Gurbuz, N., Rodriguez-Aguayo, C., Bayraktar, E., Denizli, M., Gonzalez-Villasana, V., Ivan, C., Lokesh, G. L. R., Amero, P., Catuogno, S., Haemmerle, M., Wu, S. Y., Mitra, R., Gorenstein, D. G., Volk, D. E., de Franciscis, V., Sood, A. K., Lopez-Berestein, G., Molecular therapy. Nucleic acids 2017, 9, 251-262. DOI 10.1016/j.omtn.2017.06.023.
27. Catuogno, S., Esposito, C. L., Ungaro, P., de Franciscis, V., Pharmaceuticals (Basel) 2018, 11 (3). DOI 10.3390/ph11030079.
28. Gefen, T., Castro, I., Muharemagic, D., Puplampu-Dove, Y., Patel, S., Gilboa, E., Mol Ther 2017, 25 (10), 2280-2288. DOI 10.1016/j.ymthe.2017.06.023.
29. Yoon, S., Rossi, J. J., Pharmaceuticals (Basel) 2018, 11 (3). DOI 10.3390/ph11030071.
30. Yang, S., Li, H., Xu, L., Deng, Z., Han, W., Liu, Y., Jiang, W., Zu, Y., Mol Ther Nucleic Acids 2018, 13, 164-175. DOI 10.1016/j.omtn.2018.08.023.
31. Maier, K. E., Levy, M., Mol Ther Methods Clin Dev 2016, 5, 16014. DOI 10.1038/mtm.2016.14.
32. van der Weyden, C. A., Pileri, S. A., Feldman, A. L., Whisstock, J., Prince, H. M., Blood Cancer J2017, 7 (9), e603. DOI 10.1038/bcj.2017.85.
33. Parekh, P., Kamble, S., Zhao, N., Zeng, Z., Portier, B. P., Zu, Y., Biomaterials 2013, 34 (35), 8909-8917.
34. Maier, O., Oberle, V., Hoekstra, D., Chemistry and physics of lipids 2002, 116 (1), 3-18.
35. Klymchenko, A. S., Kreder, R., Chemistry & biology 2014, 21 (1), 97-113.
36. Sezgin, E., Levental, I., Grzybek, M., Schwarzmann, G., Mueller, V., Honigmann, A., Belov, V. N., Eggeling, C., Coskun, U., Simons, K., Biochimica et Biophysica Acta (BBA)-Biomembranes 2012, 1818 (7), 1777-1784.
37. Faller, R., Biochim Biophys Acta 2016, 1858 (10), 2353-2361. DOI 10.1016/j.bbamem.2016.02.014.
38. Xiong, X., Liu, H., Zhao, Z., Altman, M. B., Lopez-Colon, D., Yang, C. J., Chang, L. J., Liu, C., Tan, W., Angewandte Chemie International Edition 2013, 52 (5), 1472-1476.
39. Lowe, E., Truscott, L. C., De Oliveira, S. N., Natural Killer Cells: Methods and Protocols 2016, 241-251.
40. Boissel, L., Betancur, M., Wels, W. S., Tuncer, H., Klingemann, H., Leukemia research 2009, 33 (9), 1255-1259.
41. Howe, S. J., Mansour, M. R., Schwarzwaelder, K., Bartholomae, C., Hubank, M., Kempski, H., Brugman, M. H., Pike-Overzet, K., Chatters, S. J., de Ridder, D., The Journal of clinical investigation 2008, 118 (9), 3143.
42. Stein, S., Ott, M. G., Schultze-Strasser, S., Jauch, A., Burwinkel, B., Kinner, A., Schmidt, M., Kramer, A., Schwable, J., Glimm, H., Nature medicine 2010, 16 (2), 198-204.
43. Braun, C. J., Bortug, K., Paruzynski, A., Witzel, M., Schwarzer, A., Rothe, M., Modlich, U., Beier, R., Gohring, G., Steinemann, D., Fronza, R., Ball, C. R., Haemmerle, R., Naundorf, S., Kuhlcke, K., Rose, M., Fraser, C., Mathias, L., Ferrari, R., Abboud, M. R., Al-Herz, W., Kondratenko, I., Marodi, L., Glimm, H., Schlegelberger, B., Schambach, A., Albert, M. H., Schmidt, M., von Kalle, C., Klein, C., Sci Transl Med 2014, 6 (227), 227ra33. DOI 10.1126/scitranslmed.3007280.
44. Simons, K., Ikonen, E., Nature 1997, 387 (6633), 569.
45. Fam, T. K., Klymchenko, A. S., Collot, M., 2018, 11 (9). DOI 10.3390/ma11091768.
46. Hincha, D. K., FEBS letters 2008, 582 (25-26), 3687-3692.

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

All such similar substitutes and modifications apparent to those skilled in the art are deemed to be within the spirit, scope and concept of the invention as defined by the appended claims.

What is claimed is:

1. A population of human NK cells adapted and configured to specifically bind to a lymphoma cell, wherein said cells comprise an aptamer that comprises, consists essentially of, or alternatively, consists of, an isolated nucleic acid sequence that encodes a tumor-specific aptamer.

2. The population of human NK cells of claim 1, wherein the aptamer comprises, consists essentially of, or alternatively, consists of, an isolated nucleic acid sequence that is at least 98% identical to the sequence of SEQ ID NO:1.

3. The population of human NK cells of claim 1, wherein the aptamer comprises, consists essentially of, or alternatively, consists of, the nucleic acid sequence of SEQ ID NO:1.

4. A method of binding a human NK cell to a mammalian cancer cell, comprising functionalizing the human NK cell to comprise one or more cell-specific aptamer sequences, such as a sequence that is at least 95% homologous to the sequence of SEQ ID NO:1, and then contacting a sample suspected of containing one or more mammalian cancer cells for a time and under conditions effective to permit the nucleic acid molecule to bind to the mammalian cancer cells, if present, in the sample.

5. The method of claim 4, further comprising means to detect the mammalian cancer cell, in vitro, in vivo, ex situ, in situ, or any combination thereof.

6. A detection kit comprising an aptamer that comprises, consists essentially of, or alternatively, consists of, the nucleic acid sequence of SEQ ID NO:1; and written instructions describing a method for their use.

7. A method for detecting the presence of human cancer cells in a sample, the method comprising contacting the sample with an oligonucleotide probe that comprises an aptamer that comprises, consists essentially of, or alternatively, consists of, the nucleic acid sequence of SEQ ID NO:1 operably linked to a first detection reagent, under conditions effective, and for a time sufficient, to detect the presence of the human cancer cells in the sample.

8. The method of claim 7, wherein the first detection reagent comprises a fluorescent label, a chromogenic label, a biotinylated label, 6-carboxyfluorescein (6-FAM), HEX, Texas Red®, Texas Red®-X, Rhodamine, Rox Reference Dye, Alexa Fluor® 488, Alexa Fluor® 584, Alexa Fluor® 633, Alexa Fluor® 660, Alexa Fluor® 680, R-phycoerythrin (R-PE), tetramethylrhodamine (TRITC), 5-carboxytetramethylrhodamine (5-TAMRA), a cyanine dye, IRDye® 800CW, ethidium bromide, or any combination thereof.

9. The method of claim 7, wherein the sample is a biological, clinical, or laboratory sample.

10. The method of claim 7, wherein the oligonucleotide probe specifically binds to one or more human lymphoma cancer cells.

11. The method of claim 7, wherein the aptamer is comprised of RNA or ssDNA.

12. The method of claim 7, wherein the oligonucleotide probe is about 20 to about 80 nucleotides in length.

13. The method of claim 12, wherein the oligonucleotide probe is about 30 to about 70 nucleotides in length.

14. The method of claim 13, wherein the oligonucleotide probe is about 40 to about 60 nucleotides in length.

15. The method of claim 7, wherein the label is detected by flow cytometry, by immunophenotyping, by tissue staining, by fluorescence microscopy, by a radionucleotide, a spin label, or by any combination thereof.

16. The method of claim 7, adapted and configured for large-scale, multi-well, microplate, or high-throughput analysis of a plurality of samples.

17. The method of claim 16, wherein the plurality of samples may be assayed simultaneously or sequentially via an automated, multi-well, microplate reader system.

18. The method of claim 17, wherein the plurality of samples includes mammalian cell culture samples, mammalian tissue culture samples, clinical isolates, one or more biological fluids of human origin, or any combination thereof.

19. A composition comprising: (1) (a) the population of human NK cells of claim 1 or (b) an aptamer that consists of an isolated nucleic acid sequence that encodes a tumor-specific aptamer; and (2) a pharmaceutically-acceptable diluent or buffer.

20. The composition of claim 19, formulated for human administration.

21. An isolated population of mammalian cells comprising an aptamer that consists of an isolated nucleic acid sequence that is at least 98% identical to the sequence of SEQ ID NO:1.

22. A therapeutic kit comprising the composition of claim 19, and a set of instructions for using the composition in the treatment or amelioration of one or more symptoms of a mammalian disease, dysfunction, impairment, injury, trauma, or abnormal metabolic condition.

23. The therapeutic kit of claim 22, wherein the composition further comprises at least one therapeutic agent.

24. A method of treating or ameliorating one or more symptoms of a disease, disorder, dysfunction, or trauma in a mammalian patient, the method comprising providing to the mammalian patient the composition of claim 19; in an amount, and for a time effective to treat or ameliorate the one or more symptoms of the disease, disorder, dysfunction or trauma in the patient.
